(19) United States
(12) Patent Application Publication
(10) Pub. No.: US 2025/0121021 A1
(43) Pub. Date: Apr. 17, 2025

(54) PREPARATION METHOD, USE, AND QUALITY DETECTION METHOD OF PANAX NOTOGINSENG-BLETILLA STRIATA ORAL LIQUID

(71) Applicant: BEIJING VVHEALTH TECHNOLOGY CO,.LTD., Beijing (CN)

(72) Inventors: Lin HAN, Beijing (CN); Nianping QIU, Beijing (CN); Ting CAI, Beijing (CN)

(73) Assignee: BEIJING VVHEALTH TECHNOLOGY CO,.LTD., Beijing (CN)

(21) Appl. No.: 18/914,406

(22) Filed: Oct. 14, 2024

(30) Foreign Application Priority Data
Oct. 12, 2023 (CN) 202311322264 .4

(51) Int. Cl. A61K 36/898; A61K 9/00; A61K 36/258; A61K 36/484; A61K 36/488; A61K 36/65; A61K 36/752; A61P 1/16

(52) U.S. Cl. CPC A61K 36/898; A61K 9/0053; A61K 36/258; A61K 36/484; A61K 36/488; A61K 36/65; A61K 36/752; A61P 1/16; A61K 2236/331; A61K 2236/39; A61K 2236/53

(57) ABSTRACT

A preparation method, a use, and a quality detection method of a Panax notoginseng-Bletilla striata oral liquid are provided. The preparation method is simple and close to the traditional Chinese medicine preparation technology. An oral liquid solution finally prepared is clear, has a high viscosity, and exhibits better efficacy than the primary drug. In the present disclosure, the prepared Panax notoginseng-Bletilla striata oral liquid is used for the first time in preparation of an antialcoholic and hepatoprotective product. Research results show that the oral liquid can enhance the oxidation resistance of endogenous antioxidase such as glutathione (GSH) to some degree, inhibit an impact of free radical-mediated lipid peroxidation on a liver injury, and exhibit a prominent antialcoholic and hepatoprotective effect for drunk mice. The quality and efficacy of an oral liquid can be monitored scientifically by the quality detection method.

### CROSS REFERENCE TO THE RELATED APPLICATIONS

This application is based upon and claims priority to Chinese Patent Application No. 202311322264.4, filed on Oct. 12, 2023, the entire contents of which are incorporated herein by reference.

### TECHNICAL FIELD

The present disclosure belongs to the technical field of preparation and detection of oral liquids of traditional Chinese medicine, and specifically relates to a preparation method, use, and quality detection method of a Panax notoginseng-Bletilla striata oral liquid.

### BACKGROUND

Excessive alcohol consumption is one of the significant triggers for a variety of diseases and harms. In the case of drinking on an empty stomach, about 60% of the alcohol is absorbed by the human body at 60 min after drinking, and about 90% to 95% of the alcohol is absorbed by the human body at 90 min to 120 min after drinking. After the alcohol enters the human body, the ingested ethanol is catalytically oxidized into acetaldehyde by alcohol dehydrogenase (ADH) and aldehyde dehydrogenase (ALDH). The acetaldehyde is further oxidized into acetic acid, and then the acetic acid further undergoes oxidative decomposition to produce carbon dioxide and water in the body. Therefore, the antialcoholic and hepatoprotective effects of a drug can be determined by measuring the activities of ADH and ALDH.

Oxidation and non-oxidation are the two major pathways for the catabolism of ethanol. In hepatocytes, ethanol is oxidized into acetaldehyde. Acetaldehyde will reduce the level of glutathione (GSH) in hepatocytes and cause significant lipid peroxidation, and the toxicity of the acetaldehyde ultimately leads to reactive oxygen species (ROS) accumulation and mitochondrial damage. Serum superoxide dismutase (SOD) can scavenge ROS in the body. Therefore, the content of GSH can reflect the degrees of oxidative stress and lipid peroxidation in the liver, and the activity of SOD in the serum is an important indicator to determine a liver injury.

Aspartate aminotransferase (AST), also known as glutamic-oxaloacetic transaminase, is mainly found in tissues such as the heart, the liver, and the skeletal muscle. When hepatocytes are slightly damaged, AST is released from the cytoplasm to the peripheral blood, and when the liver injury is further aggravated, AST in mitochondria is released into the blood, such that the level of AST in the peripheral blood is significantly increased. Clinically, the degree of liver injury is commonly assessed by detecting the activity level of AST in the peripheral blood.

The oxidative stress in hepatocytes can cause the lipid peroxidation of cell membranes to produce peroxidation products such as malondialdehyde (MDA) and ketones. MDA is a main metabolite of lipid peroxidation and is a traditional Chinese medicine biomarker for investigating lipid peroxidation and reflecting oxidative damage. An elevated level of MDA in the body indicates the presence of oxidative damage.

Total cholesterol (TC) refers to the total amount of cholesterol in all lipoproteins in the blood, including free cholesterol and cholesteryl esters. The liver is the primary organ for the synthesis and storage of various lipids. A serum concentration of TC can be used as an indicator for lipid metabolism.

Triglyceride (TG) is an organic compound produced through the esterification of three hydroxyl groups in glycerol with three fatty acid molecules. TG is a non-polar substance stored in the body in a non-hydration form. TG is an energy substance with the largest reserves and the largest production capacity in the body. The increase in TG will indirectly lead to an increase in low-density lipoprotein cholesterol.

Alanine aminotransferase (ALT) is mainly found in various cells, especially in hepatocytes. The content of ALT in the whole liver is about 100 times the content of ALT in the blood. After ethanol acts on hepatocytes for a long time, hepatocytes are damaged, and ALT is released. Therefore, the content of ALT in the serum and cell supernatant of mice in each group can be detected to reflect liver function.

In the prior art, a Panax notoginseng-Bletilla striata oral liquid and a preparation method thereof are disclosed. This Panax notoginseng-Bletilla striata oral liquid has a protective effect on gastric mucosa and liver injuries. However, the oral liquid produced by the preparation method has a low viscosity, resulting in an unsatisfactory use effect for patients.

### SUMMARY

In order to solve the above problems, the present disclosure provides a preparation method, use, and quality detection method of active ingredient for a Panax notoginseng-Bletilla striata oral liquid. The method successfully overcomes the defect of poor efficacy in the prior art, reduces the production cost and production cycle, and greatly reduces the consumption of ethanol. The preparation method is simple and close to the important traditional preparation process. An oral liquid solution finally prepared is clear, has a high viscosity, and exhibits better efficacy than the primary drug. The oral liquid is used for the first time to prepare an antialcoholic and hepatoprotective product that can play an excellent antialcoholic and hepatoprotective role.

In a first aspect, the present disclosure provides a preparation method of a Panax notoginseng-Bletilla striata oral liquid, including the following steps:
taking Bletilla striata, adding water, decocting to obtain a first decoction, filtering the first decoction, combining filtrates, and concentrating to obtain a first concentrate;
taking Panax notoginseng, Radix puerariae, Aurantii fructus immaturus, Glycyrrhiza uralensis Fisch., and Paeoniae Radix alba, adding water, decocting to obtain a second decoction, filtering the second decoction, combining filtrates, and concentrating to obtain a second concentrate;
centrifuging the first concentrate and the second concentrate separately to obtain a first centrifugate and a second centrifugate; and
mixing the first centrifugate and the second centrifugate, adding a pharmaceutically acceptable adjuvant, and adding purified water to a formulated amount.

Further, raw materials for 1,000 mL of the Panax notoginseng-Bletilla striata oral liquid include 50 g of the Bletilla striata, 8.33 g of the Panax notoginseng, 13.33 g of the Aurantii fructus immaturus, 16.67 g of the Radix puerariae, 8.33 g of the Glycyrrhiza uralensis Fisch., and 16.67 g of the Paeoniae Radix alba.

Further, the Bletilla striata is decocted in the water twice, where the water is added at an amount 10 times an amount of the Bletilla striata for a first time of decocting, the water is added at an amount 8 times the amount of the Bletilla striata for a second time of decocting, and the decocting is conducted for 1 h each time.

Further, the Panax notoginseng, the Radix puerariae, the Aurantii fructus immaturus, the Glycyrrhiza uralensis Fisch., and the Paeoniae Radix alba are decocted in the water twice, where the water is added at an amount 10 times a total mass of the Panax notoginseng, the Radix puerariae, the Aurantii fructus immaturus, the Glycyrrhiza uralensis Fisch., and the Paeoniae Radix alba for a first time of decocting, the water is added at an amount 8 times the total mass of the Panax notoginseng, the Radix puerariae, the Aurantii fructus immaturus, the Glycyrrhiza uralensis Fisch., and the Paeoniae Radix alba for a second time of decocting, and the decocting is conducted for 1.5 h each time.

Further, the centrifuging is conducted at a rotational speed of 15,000 r/min.

Further, the adjuvant includes at least one of xylitol, sodium benzoate, a fragrance, sodium cyclamate, acesulfame potassium, and citric acid.

Further, the concentrating refers to vacuum concentration at a temperature of 50° C. to 75° C. and a vacuum degree of −0.07 MPa to −0.09 MPa and further preferably −0.08 MPa.

In a second aspect, the present disclosure provides a use of the Panax notoginseng-Bletilla striata oral liquid described in the first aspect in preparation of an antialcoholic and hepatoprotective composition.

### Beneficial Effects of the Above Solution

Through an animal experiment, a drunken mouse model is constructed, an indicator for a drunkenness behavior in a test animal is observed, corresponding indexes in the serum such as AST and SOD activities and MDA and GSH contents are determined, and ADH and ALDH activities and MDA and GSH contents in the liver are determined, so as to investigate an antialcoholic function of the Panax notoginseng-Bletilla striata oral liquid and a protective effect of the Panax notoginseng-Bletilla striata oral liquid for a liver injury in drunk mice.

Results of an animal antialcoholic experiment show that mice in high-dose, medium-dose, and low-dose administration groups of the test drug all undergo phenomena such as loss of righting reflex, extension of latency time, shortening of sleep time, and shortening of sobering time, indicating that the Panax notoginseng-Bletilla striata oral liquid has a prominent protective effect for drunk mice.

Results of a hepatoprotective effect experiment show that the test drug can effectively reduce the contents of AST and MDA in the serum and the content of MDA in the liver to improve a liver injury, can effectively increase the activity of SOD and the content of GSH in the serum, and can also effectively increase the activities of ADH and ALDH and the content of GSH in the liver to reduce the liver damage and play a prominent protective role, indicating that the Panax notoginseng-Bletilla striata oral liquid has a protective effect on an acute alcoholic liver injury rat model.

In the present disclosure, an antialcoholic effect and an anti-alcoholic liver injury effect of the Panax notoginseng-Bletilla striata oral liquid are investigated from the perspective of the in vivo alcohol metabolism pathway ADH-ALDH and the detection of oxidative stress and antioxidant indicators. Through the observation of righting reflex of acutely drunk mice and the detection of MDA and GSH contents and AST and SOD activities in the serum and MDA and GSH contents and ADH and ALDH activities in the liver, it has been found that the Panax notoginseng-Bletilla striata oral liquid can prevent the drunkenness, enhance the oxidation resistance of endogenous antioxidase such as SOD and GSH to some degree, and inhibit the damage of free radical-mediated lipid peroxidation to a liver. The above study results all indicate that the Panax notoginseng-Bletilla striata oral liquid has a prominent antialcoholic and hepatoprotective effect.

In a third aspect, the present disclosure provides a quality detection method of active ingredient in the Panax notoginseng-Bletilla striata oral liquid described in the first aspect, including the following steps:
with methanol as a solvent, preparing a mixed reference solution of the active ingredient;
with methanol as a solvent, preparing a test sample solution of the Panax notoginseng-Bletilla striata oral liquid; and
determining chromatographic condition, and conducting gradient elution.

Beneficial effects of the above solution: Contents of puerarin, paeoniflorin, naringin, notoginsenoside R₁, bis(4-(glucopyranosyloxy)benzyl) 2-sec-butylmalate, ginsenoside Rg₁, and ginsenoside Rb₁ in the Panax notoginseng-Bletilla striata oral liquid are determined by quantitative analysis of multi-components by single maker, which improves a detection efficiency, has a high accuracy, and provides a basis for the quality control of the Panax notoginseng-Bletilla striata oral liquid.

In the present disclosure, with methanol as a solvent, a Panax notoginseng-Bletilla striata oral liquid test sample solution is prepared by an ultrasonic method during which components to be tested are completely dissolved. In the present disclosure, A (acetonitrile)-B (water) is adopted as a mobile phase, and stable chromatographic condition including an elution gradient, a column temperature, a detection wavelength, or the like are determined, such that detection results of all components to be tested on different instruments and chromatographic columns are stable, a relative retention time of each chromatographic peak is stable, and each chromatographic peak has a prominent peak shape and an excellent resolution.

Further, the chromatographic condition includes: a chromatographic column has a length of 150 mm to 250 mm, an inner diameter of 4.6 mm, and a particle size of 5 μm, and the number of theoretical plates is not less than 2,000 according to a peak for puerarin. Further, a process of the gradient elution includes:

### Chromatographic Column: An Octadecylsilane-Bonded Silica Gel is Adopted as a Filler.

A detection wavelength for (ginsenoside Rb₁, ginsenoside Rg₁, and notoginsenoside R₁) is 203 nm, a detection wavelength for (puerarin) is 250 nm, a detection wavelength for (paeoniflorin and bis(4-(glucopyranosyloxy)benzyl) 2-sec-butylmalate) is 230 nm, and a detection wavelength for (naringin) is 283 nm.

With a flow rate of 0.8 mL/min, acetonitrile as a mobile phase A, and water as a mobile phase B, gradient elution is conducted as follows:
0 min to 20 min: A: 10%→20%, and B: 90%→80%;
20 min to 25 min: A: 20%→24%, and B: 80%→76%;
25 min to 35 min: A: 24%→28%, and B: 76%→72%;
35 min to 50 min: A: 28%→36%, and B: 72%→64%; and
50 min to 60 min: A: 36%, and B: 64%.

Further, every 1 mL of the reference solution includes 80 μg of puerarin, 100 μg of ginsenoside Rb₁, 100 μg of ginsenoside Rg₁, 50 μg of notoginsenoside R₁, 30 μg of paeoniflorin, 150 μg of bis(4-(glucopyranosyloxy)benzyl) 2-sec-butylmalate, and 80 μg of naringin.

Further, the test sample solution is prepared by the following process:
thoroughly shaking the Panax notoginseng-Bletilla striata oral liquid, adding about 1 mL of the Panax notoginseng-Bletilla striata oral liquid to a 10 mL volumetric flask, and adding an appropriate amount of methanol to the volumetric flask; conducting an ultrasonic treatment at a power of 250 W and a frequency of 40 kHz for 30 min, and cooling; and adding methanol to a specified scale, thoroughly shaking the volumetric flask, filtering, and collecting a subsequent filtrate to obtain the test sample solution.

Further, the active ingredient includes puerarin, paeoniflorin, naringin, notoginsenoside R₁, bis(4-(glucopyranosyloxy)benzyl) 2-sec-butylmalate, ginsenoside Rg₁, and ginsenoside Rb₁.

Compared with the prior art, the above technical solutions provided in the embodiments of the present application at least have the following advantages:
1. The present disclosure discloses a preparation method of a Panax notoginseng-Bletilla striata oral liquid. Compared with a preparation method provided with reference to a positive drug (six medicinal materials are subjected to two-step extraction, where Panax notoginseng is first subjected to extraction with 70% ethanol, and then an alcohol-extraction residue and the remaining medicinal materials are added to water and decocted), the preparation method of the present disclosure adopts water extraction instead of alcohol extraction for Panax notoginseng, and conducts extraction for Bletilla striata separately. The preparation method of the present disclosure leads to a clear oral liquid with a higher viscosity than the preparation method of the positive drug. Moreover, pharmacodynamic studies have shown that the Panax notoginseng-Bletilla striata oral liquid has a comparable use effect to the primary drug, and some pharmacodynamic parameters of the Panax notoginseng-Bletilla striata oral liquid are better than those of the primary drug.
2. Compared with the preparation method of the positive drug, the preparation method of a Panax notoginseng-Bletilla striata oral liquid disclosed by the present disclosure merely adopts one extraction route (water extraction), which reduces the alcohol extraction route. That is, the preparation method provided by the present disclosure reduces a process production line, which effectively reduces a personnel cost loss and shortens a single-batch production cycle. In addition, because the alcohol extraction route is omitted in the present disclosure, the consumption of ethanol is greatly reduced. Compared with the preparation method of the positive drug, the preparation method of the present disclosure can effectively reduce the consumption of ethanol, and is more in line with the traditional preparation method of traditional Chinese medicine. Therefore, the preparation method of the present disclosure reduces a production cost and the harm to the environment and personnel, and is safety and eco-friendly.
3. A Panax notoginseng-Bletilla striata oral liquid prepared by the preparation method of a Panax notoginseng-Bletilla striata oral liquid disclosed in the present disclosure has an increased viscosity. It has been verified through a plurality of batches of pilot tests that the Panax notoginseng-Bletilla striata oral liquid has a high viscosity and a prominent clarity.
4. The present disclosure discloses a use of the Panax notoginseng-Bletilla striata oral liquid in preparation of an antialcoholic and hepatoprotective product. Through an animal experiment, a drunken mouse model is constructed, an indicator for a drunkenness behavior in a test animal is observed, corresponding indexes in the serum such as AST and SOD activities and MDA and GSH contents are determined, and ADH and ALDH activities and MDA and GSH contents in the liver are determined, so as to investigate an antialcoholic function of the Panax notoginseng-Bletilla striata oral liquid and a protective effect of the Panax notoginseng-Bletilla striata oral liquid for a liver injury in drunk mice. The study results all indicate that the Panax notoginseng-Bletilla striata oral liquid has a prominent antialcoholic and hepatoprotective effect.
5. The present disclosure discloses a quality detection method of seven active ingredient in the Panax notoginseng-Bletilla striata oral liquid. The quality detection method is easy to implement, brings practical beneficial effects to the clinical use and the quality control for the Panax notoginseng-Bletilla striata oral liquid, and is conducive to ensuring the safety, effectiveness, and quality stability of a drug.

### BRIEF DESCRIPTION OF THE DRAWINGS

The accompanying drawings here are incorporated into the specification and constitute a part of the specification, illustrate embodiments that conform to the present application, and are used together with the specification to explain the principles of the present application.

To describe the technical solutions in the embodiments of the present disclosure or in the prior art clearly, the accompanying drawings required for describing the embodiments or the prior art are briefly described below. Apparently, a person of ordinary skill in the art may still derive other accompanying drawings from these accompanying drawings without creative efforts.

FIG. 1 shows a high-performance liquid chromatography spectrum of a mixed reference of puerarin, ginsenoside Rb₁, ginsenoside Rg₁, and notoginsenoside R₁; and

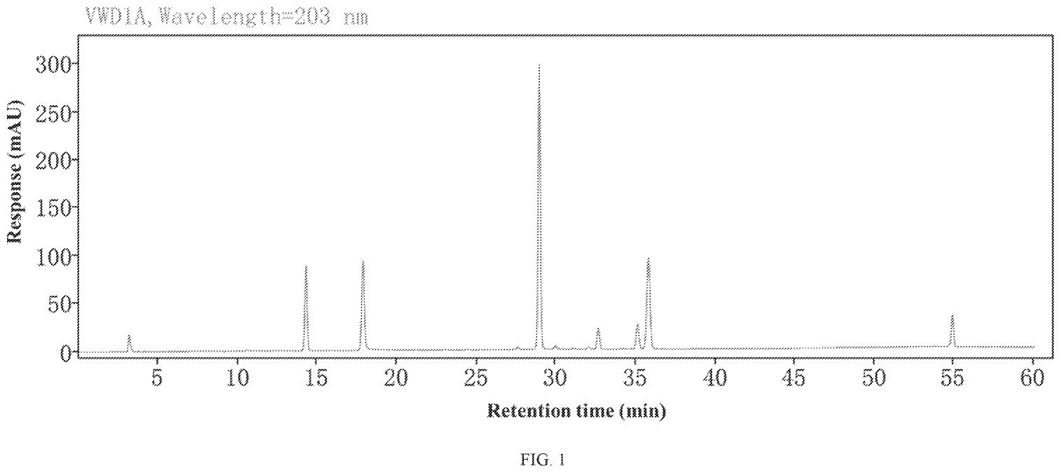

FIG. 2 is a high-performance liquid chromatography spectrum of a test sample.

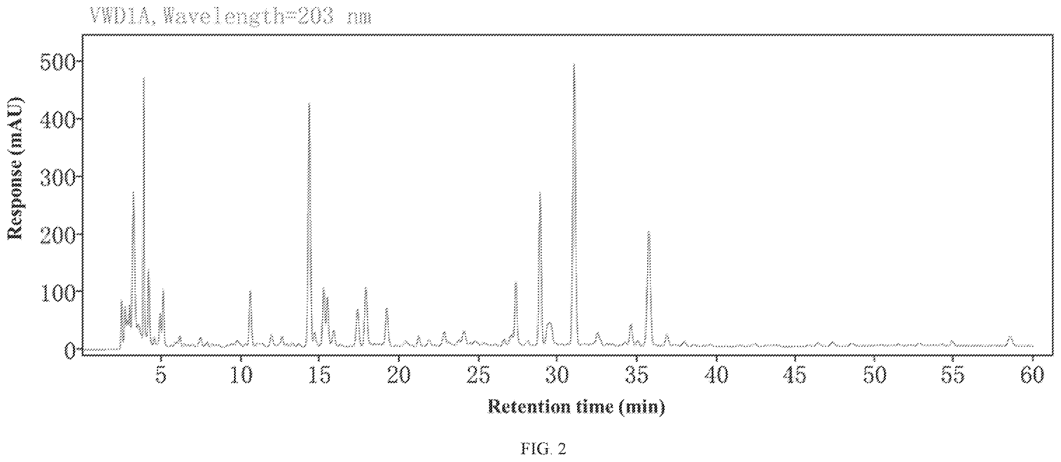

### DETAILED DESCRIPTION OF THE EMBODIMENTS

In order to make the objective, technical solutions, and advantages of the embodiments of the present application clear, the technical solutions in the embodiments of the present application are described clearly and completely below. Apparently, the described embodiments are some rather than all of the embodiments of the present application. All other embodiments obtained by those of ordinary skill in the art based on the embodiments of the present application without creative efforts should fall within the protection scope of the present application.

Unless otherwise specified, various raw materials, reagents, instruments, devices, and the like used in the present application can be purchased from the market or can be prepared by the existing methods.

The principles and features of the present disclosure are described below with reference to the embodiments. The listed embodiments are merely intended to explain the present disclosure, rather than to limit the scope of the present disclosure. If no specific conditions are specified in the embodiments, the embodiments will be implemented under conventional conditions or the conditions recommended by a manufacturer. All of the used reagents or instruments which are not specified with manufacturers are conventional commercially-available products.

### Example 1

In this example, a Panax notoginseng-Bletilla striata oral liquid was provided, including the following raw materials: 50 g of Bletilla striata, 16.67 g of Radix puerariae, 16.67 g of Paeoniae Radix alba, 13.33 g of Aurantii fructus immaturus, 8.33 g of Panax notoginseng, and 8.33 g of Glycyrrhiza uralensis Fisch.

In this example, a preparation method of the Panax notoginseng-Bletilla striata oral liquid was also provided, including the following steps:
(1) Bletilla striata was added to water, soaked for 1 h, and decocted 2 times for 1.5 h each time, where an amount of the water was 10 and 8 times an amount of the Bletilla striata for the two times of decocting, respectively. Decocted solutions each were filtered, filtrates were combined and vacuum-concentrated to obtain a concentrate 1, and the concentrate 1 was refrigerated (2° C. to 8° C.) overnight for later use.
(2) Panax notoginseng, Radix puerariae, Aurantii fructus immaturus, Glycyrrhiza uralensis Fisch., and Paeoniae Radix alba were added to water, soaked, and decocted 2 times for 1.5 h each time, where an amount of the water was 10 and 8 times an amount of the Bletilla striata for the two times of decocting, respectively. Decocted solutions each were filtered, filtrates were combined and vacuum-concentrated at a temperature of 62° C. and a vacuum degree of −0.08 MPa to obtain a concentrate 2 for later use.
(3) The concentrate 1 obtained in the step (1) was centrifuged (15,000 rpm) to obtain a centrifugate 1 for later use.
(4) The concentrate 2 obtained in the step (2) was centrifuged (15,000 rpm) to obtain a centrifugate 2 for later use.
(5) The centrifugate 1 obtained in the step (3) and the concentrate 2 obtained in the step (4) were added to a preparation tank and mixed, adjuvants such as xylitol, sodium benzoate, a fragrance, sodium cyclamate, acesulfame potassium, and citric acid were added to the preparation tank, and purified water was added to a formulated amount to obtain the Panax notoginseng-Bletilla striata oral liquid (specification: 60 mL/bottle).

### Comparative Example 1

A commercially-available Weiweikang Panax notoginseng-Bletilla striata oral liquid was provided, with a packing specification of 60 mL/bottle.

### Comparative Example 2

This comparative example was different from Example 1 in that a preparation method was as follows:
1) Medicinal materials were weighed according to the components and weights thereof in the above traditional Chinese medicine formula, and subjected to a routine pre-treatment.
2) A specified amount of Panax notoginseng was added to a multi-functional extraction tank, a first extraction was conducted for 1.5 h with 70% edible ethanol at an amount 4 to 6 times an amount of the Panax notoginseng under heating reflux, and a second extraction was conducted for 1 h with 70% edible ethanol at an amount 3 to 5 times the amount of the Panax notoginseng under heating reflux. Extraction solutions each were filtered, filtrates were combined, placed in a vacuum concentration device, heated at 55° C. to recover the ethanol, and concentrated until a relative density was 1.02 to 1.05 (50° C.) to obtain a Panax notoginseng concentrate (a Panax notoginseng residue was stored separately for later use).
3) Bletilla striata, Radix puerariae, Paeoniae Radix alba, Aurantii fructus immaturus, Glycyrrhiza uralensis Fisch., and the Panax notoginseng residue were added to a multi-functional extraction tank, and subjected to extraction with water twice for 1.5 h (the timing was started after boiling) each time, where an amount of the water was 8 to 10 times an amount of the medicinal material for a first time of extraction and an amount of the water was 6 to 8 times the amount of the medicinal material for a second time of extraction. Extraction solutions each were filtered, and filtrates were combined. The Panax notoginseng concentrate and a combined filtrate were combined in a double-effect energy-saving concentrator vacuum-concentration device and subjected to vacuum concentration at 0.06 Mpa and 75° C. until a relative density was 1.02 to 1.05 (50° C.) to obtain a concentrate. The concentrate was refrigerated in a freezer at 5±2° C. for 24 h, and a resulting supernatant was collected for later use.
4) The supernatant was added to a preparation tank, an appropriate amount of an adjuvant such as a corrigent was added to the preparation tank, stirring was conducted for dissolution, and purified water was added to 60 L. A pH was adjusted with citric acid to about 5 (a stirring time was not less than 15 min) to obtain a final preparation liquid (density: 0.98 to 1.03, 25° C.) for later use.
5) The above preparation liquid was filled into 1,000 bottles by a syrup filler, with 60 mL in each bottle.

### Test Example 1-Viscosity Determination

With reference to the detection method of a viscosity of a solution provided by the Chinese Pharmacopoeia, a viscosity of each of the oral liquids provided in Example 1 and Comparative Examples 1 to 2 was determined.

Method: 240 mL of a test sample was taken, thoroughly shaken, placed in a beaker, and tested by a viscometer for a viscosity, where rotor type: No. 1, rotational speed: 60 rpm, and when a scale of a rotor was flush with a liquid level, a viscosity value was read. In general, a constant K of a rotor-type viscometer was obtained through calibration with a standard-viscosity liquid, and thus a determination result of the rotor-type viscometer refers to a relative viscosity.

TABLE 1
Viscosity test results of 6 batches of samples in total

| Group | Batch | Batch | Batch | Batch | Batch | Batch |
|---|---|---|---|---|---|---|
| mPa · s | 1 | 2 | 3 | 4 | 5 | 6 |
| Example 1 | 7 | 7 | 8 | 6 | 7 | 8 |
| Comparative | 2 | 3 | 2 | 2 | 1 | 2 |
| Example 1 | | | | | | |
| Comparative | 1 | 1 | 1 | 1 | 1 | 1 |
| Example 2 | | | | | | |

It can be seen from Table 1 that a Panax notoginseng-Bletilla striata oral liquid prepared by the preparation method provided in the present disclosure has a high viscosity. Compared with Comparative Example 2, the preparation method provided for the positive drug (Comparative Example 1) is improved to some degree, but the sample still has a low viscosity. On the basis of the positive drug, the present application optimizes an extraction process to obtain an optimized preparation method. Results of multiple batches of verification all show that the oral liquid provided by the present disclosure has excellent process stability, and a viscosity of a sample of the oral liquid is significantly improved. The sample with an improved viscosity can increase a residence time in the stomach and increase the absorption of a drug.

In addition, in order to prove that the preparation method provided by the present disclosure does not change the efficacy, the test in Test Example 2 was conducted.

### Test Example 2-Impact on a Behavioral Indicator of Drunk Mice

In this experiment, compound methionine and choline bitartrate tablets were adopted as a positive drug. The Panax notoginseng-Bletilla striata oral liquid in Example 1 was intragastrically administered as a test sample at high, medium, and low doses, and the primary drug (the commercially-available Weiweikang Panax notoginseng-Bletilla striata oral liquid) was intragastrically administered. In a model group, distilled water was intragastrically administered at 0.20 mL/10 g. In a positive drug group, a positive control drug solution was intragastrically administered at 0.20 mL/10 g. 30 min after administration, a liquor was intragastrically administered according to the optimal liquor amount (0.17 mL/10 g) in each group. In the follow-up experimental process, the disappearance and restoration of righting reflex of mice in each group were observed and recorded.

The righting reflex, also known as righting reaction, generally refers to a reflex that occurs when an animal body is in an abnormal body position to restore a normal body position. The righting reflex was normal in sober animals, the righting reflex disappeared in drunk animals, and the righting reflex was gradually restored during a sobering process. A drunkenness latency referred to a time from the beginning of intragastric administration of the liquor to the disappearance of righting reflex in mice. A sleep time referred a time from the disappearance of righting reflex to the restoration of righting reflex once again in mice. A sobering time referred to a time from the beginning of intragastric administration of the liquor to the restoration of righting reflex once again in mice, that is, the sobering time was equal to a sum of the drunkenness latency and the sleep time. A criterion for the disappearance of righting reflex is that, when a mouse maintains a back-down posture for 30 s or more, it indicates the disappearance of righting reflex.

### 1. Experimental Materials

### 1.1 Animals

SPF-grade KM mice: which had a body weight of (20±5) g, were male, and were provided by the SiPeiFu (Beijing) Biotechnology Co., Ltd.

### 1.2 Drugs

Test sample: the Panax notoginseng-Bletilla striata oral liquid in Example 1. The oral liquid was vacuum-concentrated until a volume was ¼ of the original volume to obtain a suspension for later use, that is, the oral liquid in each bottle was vacuum-concentrated from 60 mL to 15 mL. Primary drug: the commercially-available Weiweikang Panax notoginseng-Bletilla striata oral liquid. The oral liquid was vacuum-concentrated until a volume was ¼ of the original volume to obtain a suspension for later use, that is, the oral liquid in each bottle was vacuum-concentrated from 60 mL to 15 mL.

Positive control drug: compound methionine and choline bitartrate tablets produced by the Tonghua Dongbao Pharmaceutical Co., Ltd. The compound methionine and choline bitartrate tablets were prepared with distilled water into a solution with a concentration of 0.036 g/mL.

### 1.3 Reagents and Instruments

56° Red Star Erguotou (Beijing Red Star Co., Ltd.). Wahaha purified water (Wahaha Group Co., Ltd.).

Electronic balance (Zhongshan Loease Electronic Technology Co., Ltd.). N-1300 rotary evaporator (Shanghai Ailang Instruments Co., Ltd.).

### 2. Experimental Method

### 2.1 Drunkenness Experiment

25 SPF-grade KM mice were adaptively raised for one week and then randomly divided into 5 groups with 5 mice in each group. Mice in the five groups were intragastrically administered with the 56° liquor at 0.10 mL/10 g, 0.15 mL/10 g, 0.17 mL/10 g, 0.20 mL/10 g, and 0.25 mL/10 g, respectively, and conditions of mice in each group were observed to determine the optimal liquor dose for mice.

### 2.2 Antialcoholic Experiment

In this experiment, doses of a primary drug group, a high-dose group, a medium-dose group, and a low-dose group for the Panax notoginseng-Bletilla striata oral liquid in Example 1 were set to 0.60 mL/10 g, 0.80 mL/10 g, 0.60 mL/10 g, and 0.40 mL/10 g, respectively (after concentration, actual doses of the high-dose group, the medium-dose group, and the low-dose group for the test sample were 0.15 mL/10 g, 0.20 mL/10 g, 0.15 mL/10 g, and 0.10 mL/10 g, respectively), which were 2.5 times, 3.33 times, 2.50 times, and 1.67 times the daily clinical dose for adults, respectively. In order to prevent the error impact caused by different intragastric administration amounts, in this experiment, an intragastric administration amount for each mouse was set to 0.20 mL/10 g, and if the intragastric administration amount was insufficient, distilled water was added to allow the same intragastric administration amount.

30 SPF-grade KM mice were adaptively raised for one week and then randomly divided into 5 groups according to body weights with 6 mice in each group, including a model group, a positive drug group, a primary drug group, and high-dose, medium-dose, and low-dose groups for the test sample. The mice were fasted without water deprivation for 12 h, and then the experiment was started. The test sample groups were intragastrically administered according to the high, medium, and low doses in Example 1, respectively. The primary drug group was administered according to the dose group in Example 1. The model group was intragastrically administered with distilled water at 0.20 mL/10 g. The positive drug group was intragastrically administered with a positive control solution at 0.20 mL/10 g. 30 min after administration, a liquor was intragastrically administered according to the optimal liquor amount (0.17 mL/10 g) in each group. In the follow-up experimental process, the disappearance and restoration of righting reflex of mice in each group were observed and recorded.

### 3. Experimental Results

### 3.1 Construction of a Drunkenness Model and Determination of the Optimal Liquor Dose for Test Mice

Mice in each group underwent different degrees of drunkenness after the intragastric administration of the liquor. Experimental results (Table 2) showed that the death of mice was obvious when an intragastric administration dose was 0.25 mL/10 g, and mice were not drunk at 0.10 mL/10 g which was not suitable as the optimal liquor dose. In contrast to the remaining three doses, when the intragastric administration dose for the liquor was 0.17 mL/10 g, a drunkenness time and a sobering time were moderate, and mice did not undergo death, indicating that 0.17 mL/10 g was an appropriate drunkenness-causing dose. That is, the optimal liquor dose could be set to 0.17 mL/10 g.

TABLE 2
Impacts of different liquor doses on mice

| | | | | Mor- | Drunk- | | Sober- |
|---|---|---|---|---|---|---|---|
| | Dose | | Number | tality | enness | Sleep | ing |
| | (mL/ | | of sur- | rate | latency | time | time |
| Group | 10 g) | Mice | vivors | (%) | (min) | (min) | (min) |
| 1 | 0.10 | 5 | 5 | 0 | / | / | / |
| 2 | 0.15 | 5 | 5 | 0 | 29.32 ± | 209.67 ± | 238.99 ± |
| | | | | | 8.85 | 51.60 | 46.47 |
| 3 | 0.17 | 5 | 5 | 0 | 18.88 ± | 415.00 ± | 433.88 ± |
| | | | | | 16.41 | 32.87 | 31.00 |
| 4 | 0.20 | 5 | 4 | 20 | 29.90 ± | 480.00 ± | 513.83 ± |
| | | | | | 19.51 | 0 | 20.19 |
| 5 | 0.25 | 5 | 0 | 100 | 3.52 ± | / | / |
| | | | | | 1.10 | | |

### 3.2 Antialcoholic Effect

Before the experiment, mice in each group had a smooth and neat coat, an agile response, normal righting reflex, and normal urination and defecation. For prophylactic administration, after the liquor was intragastrically administered at the optimal drunkenness-causing dose determined in 3.1, mice in each group underwent different degrees of drunkenness, which was specifically manifested as behaviors such as an excitatory state, unsteady crawling, frequent head scratching with forelimbs, and eye closing and immobility after drunkenness.

TABLE 3
Impacts on righting reflex in drunk mice (x̅ ± s, n = 6)

| Group | Drunkenness latency (min) | Sleep time (min) | Sobering time (min) |
|---|---|---|---|
| Model group | 21.22 ± 19.87 | 459.17 ± 12.24 | 480.39 ± 25.99 |
| Positive drug group | 30.40 ± 24.14 | 389.33 ± 34.55* | 419.73 ± 46.06* |
| Primary drug group | 73.75 ± 19.24**^{ΔΔ} | 349.50 ± 32.27**^{ΔΔ} | 321.58 ± 35.97**^{ΔΔ} |
| High-dose group | 89.21 ± 11.66**^{Δ} | 110.75 ± 70.23*^{Δ} | 199.96 ± 67.86**^{ΔΔ} |
| Medium-dose group | 85.09 ± 40.66**^{ΔΔ} | 232.00 ± 43.68**^{ΔΔ} | 317.09 ± 33.77**^{ΔΔ} |
| Low-dose group | 53.75 ± 31.00* | 365.00 ± 52.98 | 418.75 ± 44.98* |

Notes:
Compared with the model group,
*P < 0.05 and
**P < 0.01.
Compared with the positive drug group,
^{Δ}P < 0.05 and
^{ΔΔ}P < 0.01.

After prophylactic administration (that is, 30 min after administration, each group is intragastrically administered with the liquor), compared with the model group:
(1) Compared with the model group, drunkenness latencies of mice in the positive drug group, the primary drug group, and the high-dose, medium-dose, and low-dose administration groups for the oral liquid test sample all are prolonged. A difference between a drunkenness latency of each of the high-dose, medium-dose, and low-dose administration groups for the oral liquid test sample and a drunkenness latency of the model group is statistically significant.
(2) Compared with the model group, sleep times of mice in the positive drug group, the primary drug group, and the high-dose, medium-dose, and low-dose administration groups for the oral liquid test sample all are significantly shortened. A difference between a sleep time of each of the positive drug group and the high-dose and medium-dose administration groups and a sleep time of the model group is statistically significant.
(3) Compared with the model group, sobering times of mice in the positive drug group, the primary drug group, and the high-dose, medium-dose, and low-dose administration groups for the oral liquid test sample all are shortened. A difference between a sobering time of drunk mice in each of the positive drug group, the primary drug group, and the high-dose, medium-dose, and low-dose administration groups and a sobering time of drunk mice in the model group is statistically significant (Table 3).

When the high-dose, medium-dose, and low-dose administration groups and the primary drug group are compared with the positive drug group:
(1) Drunkenness latencies of mice in the primary drug group and the high-dose, medium-dose, and low-dose administration groups all are prolonged. A difference between a drunkenness latency of each of the primary drug group and the high-dose and medium-dose administration groups for the test sample and a drunkenness latency of the positive drug group is statistically significant.
(2) Sleep times of mice in the primary drug group and the high-dose, medium-dose, and low-dose administration groups all are shortened compared with the positive drug group. A difference between a sleep time of each of the primary drug group and the high-dose and medium-dose administration groups and a sleep time of the positive drug group is statistically significant.
(3) Sobering times of mice in the primary drug group and the high-dose, medium-dose, and low-dose administration groups all are shortened compared with the positive drug group. A difference between a sobering time of each of the primary drug group and the high-dose and medium-dose administration groups and a sobering time of the positive drug group is statistically significant.

The above experimental results show that, with the increase of a dose, a drunkenness latency of mice is gradually prolonged and a sobering time of mice is gradually shortened (Table 3). The Panax notoginseng-Bletilla striata oral liquid has a prominent antialcoholic effect for drunk mice. At a same dose, an effect of the Panax notoginseng-Bletilla striata oral liquid is comparable to or slightly better than an effect of the primary drug (Panax notoginseng-Bletilla striata oral liquid).

Test Example 3-Impacts of the Panax notoginseng-Bletilla striata oral liquid on serum SOD, MDA, AST, and GSH in drunk mice

### 1. Experimental Materials

The experimental animals and drugs were the same as those in Test Example 2.

Reagents and instruments: An AST (AST/GOT) test kit (batch No. 20221227), a micro-reduced GSH test kit (batch No. 20221226), and an MDA test kit (batch No. 20221109), which all were purchased from the Nanjing Jiancheng Institute of Biological Engineering. A total SOD activity test kit (batch No. 122921220902), which was purchased from the Beyotime Biotechnology.

Chloral hydrate (Shanghai Maclin Biochemical Technology Co., Ltd.): the chloral hydrate was prepared with distilled water into a solution with a concentration of 5% for later use. Wahaha purified water (Wahaha Group Co., Ltd.).

A high-speed refrigerated centrifuge (Thermo Fisher Scientific). A microplate reader (BioTek Instruments, Inc. in the United States). An SN-HWS-12 electric-heated thermostatic water bath (Shanghai Shangpu Instrument Equipment Co., Ltd.). An LVX-100/200 vortex shaker (Changzhou Runhua Electric Co., Ltd.). An incubator (Thermo Fisher Scientific).

### 2. Experimental Method

The experimental grouping and administration methods were the same as above. 8 h after liquor administration, mice in each group were intraperitoneally injected with 5% chloral hydrate (0.1 mL/10 g) for anaesthetization. Eyeballs were removed from mice in each group, and blood was collected, injected into a blank clean centrifuge tube, refrigerated in a refrigerator at 4° C. for 30 min, and then centrifuged at 4° C. and 3,500 r/min for 10 min to separate serum. According to instructions of each kit, SOD and AST activities and MDA and GSH contents in the serum of mice each were determined.

### 3. Experimental Results

8 h after mice were intragastrically administered with a liquor, the SOD and AST activities and MDA and GSH contents in the serum of mice in each of the administration groups and the model group were determined. Results were shown in Table 4.

TABLE 1
Impacts on serum SOD, AST, MDA, and GSH in drunk mice

| Group | SOD (unit) | AST (karmen unit) | MDA (nmol/mL) | GSH (μmol/L) |
|---|---|---|---|---|
| Model group | 0.53 ± 0.09 | 67.16 ± 14.67 | 11.15 ± 6.46 | 15.16 ± 5.25 |
| Positive drug group | 0.61 ± 0.10 | 57.00 ± 15.44 | 6.58 ± 1.75 | 35.29 ± 14.18 |
| Primary drug group | 0.75 ± 0.14** | 38.961 ± 10.65**^{Δ} | 5.12 ± 0.51 | 64.65 ± 13.41**^{Δ} |
| High-dose group | 0.96 ± 0.21**^{ΔΔ} | 31.31 ± 11.76**^{ΔΔ} | 6.98 ± 0.73 | 98.64 ± 28.39*^{Δ} |
| Medium-dose group | 0.91 ± 0.03**^{ΔΔ} | 37.40 ± 14.34**^{Δ} | 5.93 ± 1.25 | 67.65 ± 22.80* |
| Low-dose group | 0.66 ± 0.08 | 40.05 ± 15.92**^{Δ} | 4.95 ± 0.40 | 56.33 ± 7.12** |

Notes:
Compared with the model group,
*P < 0.05 and
**P < 0.01.
Compared with the positive drug group,
^{Δ}P < 0.05 and
^{ΔΔ}P < 0.01.

The results (Table 4) show that the drug in each dose group increases an SOD activity and decreases an AST activity in the serum of mice, and decreases an MDA content and increases a GSH content in the serum. Compared with the model group:
(1) SOD in the positive drug group, the primary drug group, and the high-dose, medium-dose, and low-dose groups increases compared with the model group. A difference between SOD of each of the primary drug group and the high-dose and medium-dose groups and SOD of the model group is statistically significant.
(2) AST in the positive drug group, the primary drug group, and the high-dose, medium-dose, and low-dose groups decreases compared with the model group. A difference between AST of each of the primary drug group and the high-dose, medium-dose, and low-dose groups and AST of the model group is statistically significant.
(3) MDA in the positive drug group, the primary drug group, and the high-dose, medium-dose, and low-dose groups decreases compared with the model group.
(4) GSH in the positive drug group, the primary drug group, and the high-dose, medium-dose, and low-dose groups increases compared with the model group. A difference between GSH of each of the primary drug group and the high-dose, medium-dose, and low-dose groups and GSH of the model group is statistically significant.

When the primary drug group and the high-dose, medium-dose, and low-dose groups are compared with the positive drug group:
(1) Serum SOD in the primary drug group and the high-dose, medium-dose, and low-dose groups is higher than serum SOD in the positive drug group. A difference between SOD of each of the high-dose and medium-dose groups and SOD of the positive drug group is statistically significant.
(2) Serum AST in the primary drug group and the high-dose, medium-dose, and low-dose groups is lower than serum AST in the positive drug group. A difference between AST of each of the primary drug group and the high-dose, medium-dose, and low-dose groups and AST of the positive drug group is statistically significant.
(3) Serum MDA in the primary drug group and the medium-dose and low-dose groups is lower than serum MDA in the positive drug group.
(4) Serum GSH in the primary drug group and the high-dose, medium-dose, and low-dose groups is higher than serum GSH in the positive drug group. A difference between GSH of each of the primary drug group and the high-dose group and GSH of the positive drug group is statistically significant.

The above experimental results show that the Panax notoginseng-Bletilla striata oral liquid can increase an SOD activity and decrease an AST activity in the serum of drunk mice, and can reduce an MDA content and increase a GSH content in the serum, indicating that the Panax notoginseng-Bletilla striata oral liquid can enhance the oxidation resistance of endogenous antioxidase such as SOD and GSH to some extent, inhibit the free radical-mediated lipid peroxidation damage, and exhibit a prominent antialcoholic effect for drunk mice. At a same dose, an effect of the Panax notoginseng-Bletilla striata oral liquid is comparable to or slightly better than an effect of the primary drug (Panax notoginseng-Bletilla striata oral liquid).

### Test Example 4-Impacts of the Panax notoginseng-Bletilla striata Oral Liquid on MDA, GSH, ADH, and ALDH in the Liver of Drunk Mice

### 1. Experimental Materials

The experimental animals and drugs were the same as those in Test Example 2.

Reagents: An ADH test kit (batch No. 20221226), an ALDH test kit (batch No. 20221227), a micro-reduced GSH test kit (batch No. 20221226), and an MDA test kit (batch No. 20221109), which all were purchased from the Nanjing Jiancheng Institute of Biological Engineering. A BCA protein concentration test kit (batch No. 5001101721), which was purchased from the Bolide high-tech.

Chloral hydrate and Wahaha purified water (the same as above). Normal saline (Shijiazhuang No. 4 Pharmaceutical Co., Ltd., batch No. 2206081907).

A high-speed refrigerated centrifuge (Thermo Fisher Scientific). A microplate reader (BioTek Instruments, Inc. in the United States). An SN-HWS-12 electric-heated thermostatic water bath (Shanghai Shangpu Instrument Equipment Co., Ltd.). An LVX-100/200 vortex shaker (Changzhou Runhua Electric Co., Ltd.). An incubator (Thermo Fisher Scientific). An N5000 ultraviolet-visible spectrophotometer (Shanghai Yoke Instrument Co., Ltd.). A micro-quartz cuvette (Yixing Aoruituo Optical Instrument Co., Ltd.). An IKA handheld homogenizer (Shanghai Kaiyue Electronic Technology Co., Ltd.).

### 2. Experimental Method

The experimental grouping and administration methods were the same as above. Eyeballs were removed from mice, and blood was collected. Then, the mice each were sacrificed through cervical dislocation and then quickly dissected, and a fresh liver was collected, rinsed with normal saline, and suck-dried with a filter paper. A small part of the liver was taken and prepared into a 10% liver homogenate in an ice bath, the liver homogenate was centrifuged at 4° C. and 3,000 r/min for 10 min, and a resulting supernatant was collected for later use. The ADH and ALDH activities and the MDA and GSH contents in the liver of mice were detected according to given method steps of each kit.

### 3. Experimental Results

8 h after mice were intragastrically administered with a liquor, the ADH and ALDH activities and MDA and GSH contents in the liver of mice in each of the administration groups and the model group were determined. Results were shown in Table 5.

TABLE 5
Impacts on MDA, GSH, ADH, and ALDH in the liver of drunk mice

| | MDA | GSH | ADH | ALDH |
|---|---|---|---|---|
| Group | nmol/mgprot | μmol/gprot | U/mgprot | U/mgprot |
| Model group | 1.05 ± 0.33 | 3.69 ± 0.72 | 2.25 ± 1.40 | 8.13 ± 2.07 |
| Positive drug group | 0.85 ± 0.54 | 5.67 ± 0.92* | 4.67 ± 1.60 | 9.01 ± 1.52 |
| Primary drug group | 0.46 ± 0.16 | 6.90 ± 1.71* | 8.35 ± 2.14**^{Δ} | 11.71 ± 1.65**^{Δ} |
| High-dose group | 0.47 ± 0.18 | 6.53 ± 2.37 | 6.92 ± 1.34** | 13.96 ± 1.76**^{ΔΔ} |
| Medium-dose group | 0.46 ± 0.19 | 5.87 ± 1.86 | 8.88 ± 4.35**^{ΔΔ} | 12.92 ± 1.94**^{ΔΔ} |
| Low-dose group | 0.47 ± 0.23 | 7.13 ± 1.65* | 8.92 ± 3.17**^{ΔΔ} | 11.54 ± 1.35**^{Δ} |

Notes:
Compared with the model group,
*P < 0.05 and
**P < 0.01.
Compared with the positive drug group,
^{Δ}P < 0.05 and
^{ΔΔ}P < 0.01.

The results show (Table 5) that, compared with the model group, the drug in each group decreases an MDA content and increases a GSH content and ADH and ALDH activities in the liver of mice.
(1) MDA in the liver of mice of the positive drug group, the primary drug group, and the high-dose, medium-dose, and low-dose groups tends to decrease compared with the model group.
(2) GSH in the liver of mice of the positive drug group, the primary drug group, and the high-dose, medium-dose, and low-dose groups increases compared with the model group. A difference between GSH of each of the positive drug group, the primary drug group, and the low-dose group and GSH of the model group is statistically significant.
(3) ADH in the liver of mice of the positive drug group, the primary drug group, and the high-dose, medium-dose, and low-dose groups increases compared with the model group. A difference between ADH of each of the primary drug group and the high-dose, medium-dose, and low-dose groups and ADH of the model group is statistically significant.
(4) ALDH in the liver of mice of the positive drug group, the primary drug group, and the high-dose, medium-dose, and low-dose groups increases compared with the model group. A difference between ALDH of each of the primary drug group and the high-dose, medium-dose, and low-dose groups and ALDH of the model group is statistically significant.

ALDH and ADH activities and a GSH content in the liver of mice of each of the primary drug group and the high-dose, medium-dose, and low-dose administration groups are higher than ALDH and ADH activities and a GSH content in the liver of mice of the positive drug group. An MDA content in the liver of mice of each of the primary drug group and the high-dose, medium-dose, and low-dose groups is lower than an MDA content in the liver of mice of the positive drug group:
(1) MDA in the liver of mice of the positive drug group, the primary drug group, and the high-dose, medium-dose, and low-dose groups tends to decrease compared with the model group.
(2) GSH in the liver of mice of the positive drug group, the primary drug group, and the high-dose, medium-dose, and low-dose groups tends to increase compared with the model group.
(3) ADH in the liver of mice of the positive drug group, the primary drug group, and the high-dose, medium-dose, and low-dose groups increases compared with the model group. A difference between ADH of each of the primary drug group and the medium-dose and low-dose groups and ADH of the positive drug group is statistically significant.
(4) ALDH in the liver of mice of the positive drug group, the primary drug group, and the high-dose, medium-dose, and low-dose groups increases compared with the model group. A difference between ALDH of each of the primary drug group and the high-dose, medium-dose, and low-dose groups and ALDH of the positive drug group is statistically significant.

The above experimental results show that the Panax notoginseng-Bletilla striata oral liquid can increase ADH and ALDH activities and a GSH content and decrease an MDA content in the liver of drunk mice, indicating that the Panax notoginseng-Bletilla striata oral liquid can enhance the oxidation resistance of endogenous antioxidase such as GSH to some extent, inhibit the damage of free radical-mediated lipid peroxidation to the liver, and exhibit a prominent antialcoholic and hepatoprotective effect for drunk mice. At a same dose, an effect of the Panax notoginseng-Bletilla striata oral liquid is comparable to or slightly better than an effect of the primary drug (Panax notoginseng-Bletilla striata oral liquid).

### Test Example 5-Exploration of Extraction Process Parameters

### 1. Experimental Design

A water extraction process was designed for Panax notoginseng, Radix puerariae, Aurantii fructus immaturus, Paeoniae Radix alba, and Glycyrrhiza uralensis Fisch., and an extraction ratio, puerarin, and a total of three saponins of Panax notoginseng (a total of ginsenoside Rb₁, ginsenoside Rg₁, and notoginsenoside R₁) were adopted as detection indicators. A number of extraction times was designed to be 1, 2, and 3. An extraction time was designed to be 1 h, 1.5 h, and 2 h. An added water amount was designed to be 8, 10, and 12 times. An L9 (3⁴) orthogonal test was conducted.

### 2. Experimental Method

18 parts of the following were prepared: 10 g of Panax notoginseng, 20 g of Radix puerariae, 16 g of Aurantii fructus immaturus, 20 g of Paeoniae Radix alba, and 10 g of Glycyrrhiza uralensis Fisch. were weighed, 76 g in total for each part. 18 parts of the following were prepared: 50 g of Bletilla striata was weighed. Extraction was conducted according to the factor levels and the L9 (3⁴) orthogonal test in Table 7 and Table 8. Each test was conducted 2 times in parallel. Extraction solutions each were filtered, and filtrates were combined.

### 3. Detection Method

An extraction ratio was determined as follows: 50 mL of an extract solution was taken and added to a constant-weight evaporation dish, subjected to evaporation to dryness in a water bath, and dried at 105° C. to a constant weight, and the extraction ratio was calculated. The puerarin and the total of three saponins of Panax notoginseng were detected with reference to the quality detection method of the Panax notoginseng-Bletilla striata oral liquid.

### 4. Experimental Results

TABLE 6
Extraction process factor levels

| | Factor | | |
|---|---|---|---|
| | A | | C |
| | Added water | B | Number of extraction |
| Level | amount/time | Extraction time/h | times/time |
| 1 | 8 | 1 | 1 |
| 2 | 10 | 1.5 | 2 |
| 3 | 12 | 2 | 3 |

TABLE 7
L9(3⁴) orthogonal test of the Bletilla striata extraction process

| | | Factor | | | | | |
|---|---|---|---|---|---|---|---|
| | | | | | | | Bis(4-(gluco- |
| | | | | | | Extraction | pyranosyloxy)benzyl) |
| Test No. | | A | B | C | D | ratio/% | 2-sec-butylmalate/mg |
| 1 | | 1 | 1 | 1 | 1 | 13.87 | 748.88 |
| 2 | | 1 | 2 | 2 | 2 | 21.69 | 1118.70 |
| 3 | | 1 | 3 | 3 | 3 | 25.47 | 1255.67 |
| 4 | | 2 | 1 | 2 | 3 | 21.55 | 1220.12 |
| 5 | | 2 | 2 | 3 | 1 | 24.49 | 1257.46 |
| 6 | | 2 | 3 | 1 | 2 | 14.49 | 774.87 |
| 7 | | 3 | 1 | 3 | 2 | 25.15 | 1249.09 |
| 8 | | 3 | 2 | 1 | 3 | 15.70 | 771.54 |
| 9 | | 3 | 3 | 2 | 1 | 22.04 | 1124.26 |
| Extraction | K₁ | 20.34 | 20.19 | 14.69 | 20.13 | | |
| ratio | K₂ | 20.18 | 20.63 | 21.76 | 20.44 | | |
| | K₃ | 20.96 | 20.67 | 25.04 | 20.91 | | |
| | R | 0.79 | 0.48 | 10.35 | 0.77 | | |
| Bis(4-(gluco- | K₁ | 1041.08 | 1072.70 | 765.10 | 1043.53 | | |
| pyranosyloxy)benzyl) | K₂ | 1084.15 | 1049.23 | 1154.36 | 1047.55 | | |
| 2-sec-butylmalate | K₃ | 1048.30 | 1051.60 | 1254.07 | 1082.44 | | |
| | R | 43.07 | 23.46 | 488.98 | 38.91 | | |

TABLE 8
Analysis of variance of the Bletilla striata
extraction process (extraction ratio)

| Source of | Sum of squares of | Degree of | | | |
|---|---|---|---|---|---|
| variance | deviations | freedom | Mean square | F value | P |
| A | 1.03 | 2.00 | 0.52 | 1.13 | 0.469 |
| B | 0.42 | 2.00 | 0.21 | 0.46 | 0.684 |
| C | 167.89 | 2.00 | 83.95 | 184.73 | 0.005 |
| Error | 0.91 | 2.00 | 0.45 | | |

TABLE 9
Analysis of variance of the Bletilla striata extraction process
(Bis(4-(glucopyranosyloxy)benzyl) 2-sec-butylmalate)

| Source of | Sum of squares of | Degree of | | | |
|---|---|---|---|---|---|
| variance | deviations | freedom | Mean square | F value | P |
| A | 3192.23 | 2.00 | 1596.12 | 1.16 | 0.463 |
| B | 1001.20 | 2.00 | 500.60 | 0.36 | 0.733 |
| C | 400566.87 | 2.00 | 200283.44 | 145.80 | 0.007 |
| Error | 2747.46 | 2.00 | 1373.73 | | |

TABLE 10
L9(3⁴) orthogonal test of the extraction process for the remaining medicinal materials

| | | Factor | | | | | | |
|---|---|---|---|---|---|---|---|---|
| | | | | | | | | Three saponins |
| | | | | | | Extraction | | of Panax |
| Test No. | | A | B | C | D | ratio/% | Puerarin/mg | notoginseng/mg |
| 1 | | 1 | 1 | 1 | 1 | 17.75 | 402.14 | 475.20 |
| 2 | | 1 | 2 | 2 | 2 | 26.46 | 528.50 | 545.89 |
| 3 | | 1 | 3 | 3 | 3 | 29.87 | 544.78 | 586.83 |
| 4 | | 2 | 1 | 2 | 3 | 26.50 | 506.34 | 580.59 |
| 5 | | 2 | 2 | 3 | 1 | 29.68 | 558.32 | 584.30 |
| 6 | | 2 | 3 | 1 | 2 | 20.64 | 429.34 | 444.81 |
| 7 | | 3 | 1 | 3 | 2 | 27.94 | 567.32 | 558.26 |
| 8 | | 3 | 2 | 1 | 3 | 21.41 | 450.10 | 483.22 |
| 9 | | 3 | 3 | 2 | 1 | 28.11 | 563.24 | 571.72 |
| Extraction | K₁ | 24.7 | 24.1 | 19.9 | 25.2 | | | |
| ratio | K₂ | 25.6 | 25.9 | 27.0 | 25.0 | | | |
| | K₃ | 25.8 | 26.2 | 29.2 | 25.9 | | | |
| | R | 1.1 | 2.1 | 9.2 | 0.9 | | | |
| Puerarin | K₁ | 491.8 | 491.9 | 427.2 | 507.9 | | | |
| | K₂ | 498.0 | 512.3 | 532.7 | 508.4 | | | |
| | K₃ | 526.9 | 512.5 | 556.8 | 500.4 | | | |
| | R | 35.1 | 20.5 | 129.6 | 8.0 | | | |
| Three | K₁ | 536.0 | 538.0 | 467.7 | 543.7 | | | |
| saponins of | K₂ | 536.6 | 537.8 | 566.1 | 516.3 | | | |
| Panax | K₃ | 537.7 | 534.5 | 576.5 | 550.2 | | | |
| notoginseng | R | 1.8 | 3.6 | 108.7 | 33.9 | | | |

TABLE 11
Analysis of variance of the extraction process (extraction ratio)

| Source of | Sum of squares of | Degree of | | | |
|---|---|---|---|---|---|
| variance | deviations | freedom | Mean square | F value | P |
| A | 2.15 | 2.00 | 1.07 | 1.51 | 0.398 |
| B | 7.91 | 2.00 | 3.96 | 5.57 | 0.152 |
| C | 140.04 | 2.00 | 70.02 | 98.66 | 0.010 |
| Error | 1.42 | 2.00 | 0.71 | | |

TABLE 12
Analysis of variance of the extraction process (puerarin)

| Source of | Sum of squares of | Degree of | | | |
|---|---|---|---|---|---|
| variance | deviations | freedom | Mean square | F value | P |
| A | 2103.40 | 2 | 1051.70 | 17.519 | 0.054 |
| B | 836.17 | 2 | 418.08 | 6.964 | 0.126 |
| C | 28511.32 | 2 | 14255.66 | 237.462 | 0.004 |
| Error | 120.07 | 2 | 60.03 | | |

TABLE 13
Analysis of variance of the extraction process
(three saponins of Panax notoginseng)

| Source of | Sum of squares of | Degree of | | | |
|---|---|---|---|---|---|
| variance | deviations | freedom | Mean square | F value | P |
| A | 4.81 | 2 | 2.41 | 0.002 | 0.998 |
| B | 23.97 | 2 | 11.98 | 0.012 | 0.988 |
| C | 21595.61 | 2 | 10797.80 | 11.117 | 0.083 |
| Error | 1942.52 | 2 | 971.26 | | |

### 5. Experimental Result Analysis

(1) Result Analysis of the Bletilla striata Extraction Process

The results in Table 7 and Table 8 show that an order of impacts of the factors on a Bletilla striata extraction ratio is C>A>B. The analysis of variance shows that there is a statistical difference in terms of the impact of the factor C on a yield, and there is no statistical difference in terms of the impacts of the factors A and B on a yield. The results in Table 7 and Table 9 show that an order of the factors on bis(4-(glucopyranosyloxy)benzyl) 2-sec-butylmalate is C>A>B. Results of the analysis of variance show that there is a statistical difference in terms of the impact of C on a yield of bis(4-(glucopyranosyloxy)benzyl) 2-sec-butylmalate, and there is no statistical difference in terms of the impacts of the factors A and B on bis(4-(glucopyranosyloxy)benzyl) 2-sec-butylmalate.

According to the comprehensive analysis of the above results, the optimal extraction process is A2B2C3 where extraction is conducted 3 times for 1.5 h each time and an amount of added water each time is 10 times an amount of Bletilla striata. Given that the increase of one extraction time leads to the increase of a cost and does not match the increase of an extraction efficiency, the sub-optimal extraction process is A2B2C2 where extraction is conducted 2 times for 1.5 h each time, water is added at an amount 10 times an amount of Bletilla striata for a first time of extraction, and water is added at an amount 8 times the amount of Bletilla striata for a second time of extraction.

### (2) Result Analysis of the Extraction Process for the Remaining Medicinal Materials

With a yield as an indicator, it can be seen from Table 10 and Table 11 that an order of impacts of the factors on the yield is C>B>A. The analysis of variance shows that there is a statistical difference in terms of the impact of the factor C on the yield, and there is no statistical difference in terms of the impacts of the factors A and B on the yield.

With puerarin as an indicator, it can be seen from Table 10 and Table 12 that an order of impacts of the factors is C>A>B. The analysis of variance shows that there is a statistical difference in terms of the impact of the factor C on puerarin extraction.

With a total of three saponins of Panax notoginseng as an indicator, it can be seen from Table 10 and Table 13 that an order of impacts of the factors is C>B>A. The analysis of variance shows that there is no statistical difference in terms of the impacts of the factors A, B, and C on the extraction of the saponins of Panax notoginseng.

According to the comprehensive analysis of the above results, the optimal extraction process is A2B2C3 where extraction is conducted 3 times for 1.5 h each time and an amount of added water each time is 10 times an amount of the remaining medicinal materials. Given that the increase of one extraction time leads to the increase of a cost and does not match the increase of an extraction efficiency, the sub-optimal extraction process is A2B2C2 where extraction is conducted 2 times for 1.5 h each time, water is added at an amount 10 times an amount of the remaining medicinal materials for a first time of extraction, and water is added at an amount 8 times the amount of the remaining medicinal materials for a second time of extraction.

### 6. Verification of the Extraction Processes

(1) Verification of the Bletilla striata Extraction Process

The above optimal process and the sub-optimal process each were verified by the following process: 50 g of Bletilla striata was weighed. In the optimal process, decoction was conducted 3 times for 1.5 h with water at an amount 10 times an amount of Bletilla striata each time. In the sub-optimal process, decoction was conducted 2 times for 1.5 h each time, where water is added at an amount 10 times the amount of Bletilla striata for a first time and water is added at an amount 8 times the amount of Bletilla striata for a second time. An extraction solution was filtered to obtain a medicinal liquid, and a content of each component in the medicinal liquid was determined.

TABLE 14
Verification of the Bletilla striata extraction processes

| | | Extraction | Bis(4-(glucopyranosyloxy)benzyl) |
|---|---|---|---|
| Test | | ratio/% | 2-sec-butylmalate/mg |
| Optimal process | 1 | 24.60 | 1245.08 |
| | 2 | 24.42 | 1258.71 |
| | Mean | 24.51 | 1251.90 |
| | value | | |
| Sub-optimal | 3 | 21.63 | 1129.09 |
| process | 4 | 21.77 | 1121.95 |
| | Mean | 21.70 | 1125.52 |
| | value | | |

The results show that the sub-optimal process has a high extraction efficiency, is not significantly different from the optimal process in terms of an extraction ratio and a bis(4-(glucopyranosyloxy)benzyl) 2-sec-butylmalate content, and is stable and feasible. In order to reduce a cost, the sub-optimal process is selected as a production process of the product. That is, extraction is conducted 2 times for 1.5 h each time, where water is added at an amount 10 times an amount of Bletilla striata for a first time and water is added at an amount 8 times the amount of Bletilla striata for a second time.

### (2) Verification of the Extraction Process for the Remaining Medicinal Materials

The above-mentioned optimal conditions and sub-optimal process were verified by the following process: 20 g of Radix puerariae, 10 g of Panax notoginseng (crushed), 20 g of Paeoniae Radix alba, 16 g of Aurantii fructus immaturus, and 10 g of Glycyrrhiza uralensis Fisch. were weighed, 76 g in total. In the optimal process, decoction was conducted 3 times for 1.5 h with water at an amount 10 times an amount of the remaining medicinal materials each time. In the sub-optimal process, decoction was conducted 2 times for 1.5 h each time, where water is added at an amount 10 times the amount of the remaining medicinal materials for a first time and water is added at an amount 8 times the amount of the remaining medicinal materials for a second time. An extraction solution was filtered to obtain a medicinal liquid, and a content of each component in the medicinal liquid was determined.

TABLE 15
Verification of the extraction processes

| | | Extraction | Total | Three saponins in |
|---|---|---|---|---|
| Test | | ratio/% | puerarin/mg | Panax notoginseng/mg |
| Optimal | 1 | 29.72 | 556.35 | 579.63 |
| process | 2 | 29.54 | 569.77 | 567.28 |
| | Mean | 29.26 | 563.06 | 573.46 |
| | value | | | |
| Sub-optimal | 3 | 27.89 | 557.97 | 559.17 |
| process | 4 | 27.68 | 560.69 | 565.39 |
| | Mean | 27.79 | 559.33 | 562.28 |
| | value | | | |

The results show that there is no significant difference between the optimal process and the sub-optimal process in terms of an extraction ratio, total puerarin, and a total of three saponins in Panax notoginseng, and the extraction processes are stable and feasible. In order to reduce a cost, the sub-optimal process is selected as an extraction process of the product. That is, extraction is conducted 2 times for 1.5 h each time, where water is added at an amount 10 times an amount of the remaining medicinal materials for a first time and water is added at an amount 8 times the amount of the remaining medicinal materials for a second time.

### 7. Preparation Process

An oral liquid was prepared with reference to Example 1, which was different from Comparative Example 1 in that: Extraction solutions obtained by the two extraction routes were concentrated separately, where a concentration temperature for a Bletilla striata extraction solution was lower than or equal to 75° C. Resulting concentrates were allowed to stand overnight, centrifuged, and then mixed, adjuvants were added and dissolved, and purified water was added to a formulated amount to obtain an oral liquid that was slightly bitter and viscous.

The above are only specific implementations of the present application, which allows those skilled in the art to understand or implement the present application. Various modifications to the embodiments are readily apparent to a person skilled in the art, and the generic principles defined herein may be practiced in other embodiments without departing from the spirit or scope of the present application. Thus, the present application is not limited to the embodiments shown herein, but falls within the widest scope consistent with the principles and novel features disclosed herein.

What is claimed is:

1. A preparation method of a Panax notoginseng-Bletilla striata oral liquid, comprising the following steps:
taking Bletilla striata, adding first water, decocting to obtain a first decoction, filtering the first decoction, combining a first filtrate, and concentrating to obtain a first concentrate;
taking Panax notoginseng, Radix puerariae, Aurantii fructus immaturus, Glycyrrhiza uralensis Fisch., and Paeoniae Radix alba, adding second water, decocting to obtain a second decoction, filtering the second decoction, combining a second filtrate, and concentrating to obtain a second concentrate;
centrifuging the first concentrate and the second concentrate separately to obtain a first centrifugate and a second centrifugate; and
mixing the first centrifugate and the second centrifugate, adding a pharmaceutically acceptable adjuvant, and adding purified water to a formulated amount, to obtain the Panax notoginseng-Bletilla striata oral liquid.

2. The preparation method of the Panax notoginseng-Bletilla striata oral liquid according to claim 1, wherein raw materials for 1,000 mL of the Panax notoginseng-Bletilla striata oral liquid comprise 50 g of the Bletilla striata, 8.33 g of the Panax notoginseng, 13.33 g of the Aurantii fructus immaturus, 16.67 g of the Radix puerariae, 8.33 g of the Glycyrrhiza uralensis Fisch., and 16.67 g of the Paeoniae Radix alba.

3. The preparation method of the Panax notoginseng-Bletilla striata oral liquid according to claim 1, wherein the Bletilla striata is decocted in the first water twice, wherein the first water is added at an amount 10 times a mass of the Bletilla striata for a first time of the decocting, the first water is added at an amount 8 times the mass of the Bletilla striata for a second time of the decocting, and the decocting is conducted for 1 h each time.

4. The preparation method of the Panax notoginseng-Bletilla striata oral liquid according to claim 1, wherein the Panax notoginseng, the Radix puerariae, the Aurantii fructus immaturus, the Glycyrrhiza uralensis Fisch., and the Paeoniae Radix alba are decocted in the second water twice, wherein the second water is added at an amount 10 times a total mass of the Panax notoginseng, the Radix puerariae, the Aurantii fructus immaturus, the Glycyrrhiza uralensis Fisch., and the Paeoniae Radix alba for a first time of the decocting, the second water is added at an amount 8 times the total mass of the Panax notoginseng, the Radix puerariae, the Aurantii fructus immaturus, the Glycyrrhiza uralensis Fisch., and the Paeoniae Radix alba for a second time of the decocting, and the decocting is conducted for 1.5 h each time.

5. The preparation method of the Panax notoginseng-Bletilla striata oral liquid according to claim 1, wherein the centrifuging is conducted at a rotational speed of 15,000 r/min.

6. The preparation method of the Panax notoginseng-Bletilla striata oral liquid according to claim 1, wherein the pharmaceutically acceptable adjuvant comprises at least one of xylitol, sodium benzoate, a fragrance, sodium cyclamate, acesulfame potassium, and citric acid.

7. The preparation method of the Panax notoginseng-Bletilla striata oral liquid according to claim 1, wherein the concentrating refers to a vacuum concentration at a temperature of 50° C. to 75° C. and a vacuum degree of −0.07 MPa to −0.09 MPa.

8. A preparation method of an antialcoholic and hepatoprotective composition, comprising using a Panax notoginseng-Bletilla striata oral liquid prepared by the preparation method according to claim 1.

9. A quality detection method of an active ingredient in a Panax notoginseng-Bletilla striata oral liquid, comprising the following steps:
with methanol as a first solvent, preparing a mixed reference solution of the active ingredient;
with methanol as a second solvent, preparing a test sample solution of the Panax notoginseng-Bletilla striata oral liquid; and
determining a chromatographic condition, and conducting a gradient elution.

10. The quality detection method of the active ingredient in the Panax notoginseng-Bletilla striata oral liquid according to claim 9, wherein the active ingredient comprises puerarin, paeoniflorin, naringin, notoginsenoside R₁, bis(4-(glucopyranosyloxy)benzyl) 2-sec-butylmalate, ginsenoside Rg₁, and ginsenoside Rb₁.

11. The preparation method of the Panax notoginseng-Bletilla striata oral liquid according to claim 2, wherein the Bletilla striata is decocted in the first water twice, wherein the first water is added at an amount 10 times a mass of the Bletilla striata for a first time of the decocting, the first water is added at an amount 8 times the mass of the Bletilla striata for a second time of the decocting, and the decocting is conducted for 1 h each time.

12. The preparation method of the Panax notoginseng-Bletilla striata oral liquid according to claim 2, wherein the Panax notoginseng, the Radix puerariae, the Aurantii fructus immaturus, the Glycyrrhiza uralensis Fisch., and the Paeoniae Radix alba are decocted in the second water twice, wherein the second water is added at an amount 10 times a total mass of the Panax notoginseng, the Radix puerariae, the Aurantii fructus immaturus, the Glycyrrhiza uralensis Fisch., and the Paeoniae Radix alba for a first time of the decocting, the second water is added at an amount 8 times the total mass of the Panax notoginseng, the Radix puerariae, the Aurantii fructus immaturus, the Glycyrrhiza uralensis Fisch., and the Paeoniae Radix alba for a second time of the decocting, and the decocting is conducted for 1.5 h each time.

13. The preparation method of the antialcoholic and hepatoprotective composition according to claim 8, wherein in the preparation method of the Panax notoginseng-Bletilla striata oral liquid, raw materials for 1,000 mL of the Panax notoginseng-Bletilla striata oral liquid comprise 50 g of the Bletilla striata, 8.33 g of the Panax notoginseng, 13.33 g of the Aurantii fructus immaturus, 16.67 g of the Radix puerariae, 8.33 g of the Glycyrrhiza uralensis Fisch., and 16.67 g of the Paeoniae Radix alba.

14. The preparation method of the antialcoholic and hepatoprotective composition according to claim 8, wherein in the preparation method of the Panax notoginseng-Bletilla striata oral liquid, the Bletilla striata is decocted in the first water twice, wherein the first water is added at an amount 10 times a mass of the Bletilla striata for a first time of the decocting, the first water is added at an amount 8 times the mass of the Bletilla striata for a second time of the decocting, and the decocting is conducted for 1 h each time.

15. The preparation method of the antialcoholic and hepatoprotective composition according to claim 8, wherein in the preparation method of the Panax notoginseng-Bletilla striata oral liquid, the Panax notoginseng, the Radix puerariae, the Aurantii fructus immaturus, the Glycyrrhiza uralensis Fisch., and the Paeoniae Radix alba are decocted in the second water twice, wherein the second water is added at an amount 10 times a total mass of the Panax notoginseng, the Radix puerariae, the Aurantii fructus immaturus, the Glycyrrhiza uralensis Fisch., and the Paeoniae Radix alba for a first time of the decocting, the second water is added at an amount 8 times the total mass of the Panax notoginseng, the Radix puerariae, the Aurantii fructus immaturus, the Glycyrrhiza uralensis Fisch., and the Paeoniae Radix alba for a second time of the decocting, and the decocting is conducted for 1.5 h each time.

16. The preparation method of the antialcoholic and hepatoprotective composition according to claim 8, wherein in the preparation method of the Panax notoginseng-Bletilla striata oral liquid, the centrifuging is conducted at a rotational speed of 15,000 r/min.

17. The preparation method of the antialcoholic and hepatoprotective composition according to claim 8, wherein in the preparation method of the Panax notoginseng-Bletilla striata oral liquid, the pharmaceutically acceptable adjuvant comprises at least one of xylitol, sodium benzoate, a fragrance, sodium cyclamate, acesulfame potassium, and citric acid.

18. The preparation method of the antialcoholic and hepatoprotective composition according to claim 8, wherein in the preparation method of the Panax notoginseng-Bletilla striata oral liquid, the concentrating refers to a vacuum concentration at a temperature of 50° C. to 75° C. and a vacuum degree of −0.07 MPa to −0.09 MPa.
